(19) United States
(12) Patent Application Publication
(10) Pub. No.: US 2024/0293541 A1
(43) Pub. Date: Sep. 5, 2024

(54) LSP1-DEFICIENT T CELL

(71) Applicant: THE CATHOLIC UNIVERSITY OF KOREA INDUSTRY-ACADEMIC COOPERATION FOUNDATION, Seoul (KR)

(72) Inventors: Wan-Uk KIM, Seoul (KR); Riri KWON, Incheon (KR); Naeun LEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/564,370

(22) PCT Filed: May 26, 2021
(86) PCT No.: PCT/KR2021/006538

§ 371 Date: Nov. 27, 2023

(30) Foreign Application Priority Data
May 26, 2020 (KR) 10-2020-0063204

(51) Int. Cl. A61K 39/00; A61K 39/395; A61P 35/00

(52) U.S. Cl. CPC A61K 39/4611; A61K 39/3955; A61K 39/4637; A61P 35/00; A61K 2039/505; A61K 2039/585; A61K 2239/39; A61K 2239/57

(57) ABSTRACT

The present invention relates to a Leukocyte-specific protein 1 (LSP1)-deficient T cell-based anticancer immunotherapy. According to the present invention, tumor growth, tumor volume and size were reduced by means of LSP1 knockout, and LSP1 deficiency in T cells increases the number, distribution frequency, migration, and invasion of T cells, and increases the production of an anti-tumor cytokine to inhibit tumor growth and enhance tumor-killing ability. Thus, the present invention can be used for T cell-based immunotherapy.

### TECHNICAL FIELD

The present invention relates to a Leukocyte-specific protein 1 (LSP1)-deficient T cell-based anticancer immunotherapy.

### BACKGROUND ART

Despite numerous advances in medical research, cancer remains the leading cause of death throughout the civilized world. Non-specific approach to cancer treatment, such as surgery, radiotherapy, and generalized chemotherapy, has been successful in treating a selective group of circulating and slow-growing solid cancer. However, many solid tumors are highly resistant to this approach, and in this case, the prognosis is very poor. Standard therapy includes radiation therapy or chemotherapy after cell resection, but cure is difficult and recurrences are frequent. An emerging field in such cancer therapy is immunotherapy. A general principle is to impart the ability to deploy actually rejection reactions to a patient to be treated, especially malignant cells. There are developing numerous immunological strategies including 1. adoptive immunotherapy using various types of stimulated autologous cells; 2. systemic transfer of allogenic lymphocytes; 3. intratumoral implantation of immunologically reactive cells; and 4. vaccination at a distant site to generate a systemic-tumor-specific immune response. Among these, adoptive transfer of tumor antigen-specific lymphocytes is promising as the adoptive transfer immunotherapy, but to date, these attempts have been mainly based on mononuclear cells from peripheral blood vessels or tumor infiltrating lymphocytes (TILs) from new tumor subjects. In a recent treatment trial for patients with malignant melanoma, self-transfer treatment attempts of proliferated TILs were reported to have an objective response rate of up to 51%. The TIL cells are few in number and frequently become anergic due to an immunosuppressive mechanism from the tumor, so that it takes a long period of time (several months) for their proliferation.

An immune contexture consists of the density, composition, and functional condition of tumor-infiltrating leukocytes (TILs) determines tumor progression and efficacy of anti-tumor immunotherapy, including antibody (Ab)-based immunotherapy against programmed cell death protein 1 (PD-1) (Fridman W H, Zitvogel L, Sautes-Fridman C, et al. Nat Rev Clin Oncol 2017; 14(12):717-34.). Several studies have reported that high density of T cells has a positive correlation between favorable prognosis and survival in patients with various cancers, including colon cancer, non-small cell lung cancer, hepatocellular carcinoma, pancreatic cancer, gastric cancer, and melanoma. Accordingly, adoptive transfer using antigen-activated T cells, especially chimeric antigen receptor (CAR)-T cells, has emerged as one of the promising strategies to improve the efficacy of anticancer therapy (Lim W A, June C H. Cell 2017; 168(4):724-40.). For example, CD19-targeted CAR-T cell therapy had significantly high improvement in patients with hematologic malignancies, including relapsed or refractory B-cell acute lymphoblastic leukemia and lymphoma.

Despite success in hematological malignancies, CAR-T cell therapy has not always been effective and has been disappointing in solid tumors. One of the major obstacles to such T-cell-based cancer immunotherapy may be insufficient trafficking of T cells to the tumor mass. In clinical and preclinical studies, despite infusion of large amounts of T cells after ex vivo expansion, only a small amount of the transferred T cells may reach the interior of the tumor tissue (Slaney C Y, Kershaw M H, Darcy P K. Cancer Res 2014; 74(24):7168-74.). It is not yet known why trafficking, infiltration, and invasion of T cells are insufficient, but the reason is that the shape of solid tumors is more fibrotic and less invasive, ultimately establishing an immunosuppressive tumor environment (TME). Therefore, to maximize the effect of T-cell-based immunotherapy for solid tumors, there is a need to develop methods capable of successfully transferring immunocompetent T cells inside the tumor mass by destroying or bypassing the fibrotic and immunosuppressive TME.

### DISCLOSURE

### Technical Problem

An object of the present invention is to provide a pharmaceutical composition for preventing or treating cancer.

Another object of the present invention is to provide LSP1 gene-deleted T cells.

Yet another object of the present invention is to provide an anticancer adjuvant.

Yet another object of the present invention is to provide a method for providing information for predicting the prognosis of cancer.

Yet another object of the present invention is to provide a method for predicting an anticancer therapeutic effect.

Yet another object of the present invention is to provide a pharmaceutical composition for preventing or treating cancer caused by LSP1 overexpression including an LSP1 inhibitor as an active ingredient.

Yet another object of the present invention is to provide a pharmaceutical composition for preventing or treating cancer caused by LSP1 overexpression including LSP1 gene-deleted T cells as an active ingredient.

Yet another object of the present invention is to provide a method for preventing or treating cancer including administering a pharmaceutical composition including a Leukocyte-specific protein 1 (LSP1) expression inhibitor as an active ingredient to a subject.

Yet another object of the present invention is to provide a method for preventing or treating cancer including administering a pharmaceutical composition including LSP1 gene-deleted T cells as an active ingredient to a subject.

Yet another object of the present invention is to provide a method for preventing or treating cancer caused by LSP1 overexpression including administering a pharmaceutical composition including an LSP1 inhibitor to a subject.

Yet another object of the present invention is to provide a method for preventing or treating cancer caused by LSP1 overexpression including administering a pharmaceutical composition including LSP1 gene-deleted T cells as an active ingredient to a subject.

### Technical Solution

One aspect of the present invention provides a pharmaceutical composition for preventing or treating cancer including an LSP1 expression inhibitor as an active ingredient.

Another aspect of the present invention provides LSP1 gene-deleted T cells.

Yet another aspect of the present invention provides a pharmaceutical composition for preventing or treating cancer including LSP1 gene-deleted T cells as an active ingredient.

Yet another aspect of the present invention provides an anticancer adjuvant for enhancing the activity of an anticancer agent including an LSP1 expression inhibitor or LSP1 gene-deleted T cells as an active ingredient.

Yet another aspect of the present invention provides a method for providing information for predicting the prognosis of cancer.

Yet another aspect of the present invention provides a method for predicting an anticancer therapeutic effect.

Yet another aspect of the present invention provides a pharmaceutical composition for preventing or treating cancer caused by LSP1 overexpression including an LSP1 inhibitor as an active ingredient.

Yet another aspect of the present invention provides a pharmaceutical composition for preventing or treating cancer caused by LSP1 overexpression including LSP1 gene-deleted T cells as an active ingredient.

Yet another aspect of the present invention provides a method for preventing or treating cancer including administering a pharmaceutical composition including a Leukocyte-specific protein 1 (LSP1) expression inhibitor as an active ingredient to a subject.

Yet another aspect of the present invention provides a method for preventing or treating cancer including administering a pharmaceutical composition including LSP1 gene-deleted T cells as an active ingredient to a subject.

Yet another aspect of the present invention provides a method for preventing or treating cancer caused by LSP1 overexpression including administering a pharmaceutical composition including an LSP1 inhibitor as an active ingredient to a subject.

Yet another aspect of the present invention provides a method for preventing or treating cancer caused by LSP1 overexpression including administering a pharmaceutical composition including LSP1 gene-deleted T cells as an active ingredient to a subject.

### Advantageous Effects

According to the present invention, it was confirmed that tumor growth, tumor volume and size were reduced by means of LSP1 knockout. In addition, LSP1 deficiency in T cells increases the number, distribution frequency, migration, and invasion of T cells, and increases the production of an anti-tumor cytokine to inhibit tumor growth and enhance tumor-killing ability. Therefore, the present invention can be used as a target for T cell-based immunotherapy.

### DESCRIPTION OF DRAWINGS

FIG. 1A is a diagram confirming changes in melanoma growth in a tumor caused by Lsp1 deficiency.

FIG. 1B is a diagram confirming changes in melanoma volume in a tumor caused by Lsp1 deficiency.

FIG. 1C is a diagram confirming changes in melanoma weight in a tumor caused by Lsp1 deficiency.

FIG. 1D is a diagram confirming changes in the number of TILs in a tumor caused by Lsp1 deficiency.

FIG. 1E is a diagram confirming the infiltration of CD4⁺ T cells in a tumor caused by Lsp1 deficiency by immunohistochemical staining.

FIG. 1F is a diagram confirming the infiltration of CD8⁺ T cells in a tumor caused by Lsp1 deficiency by immunohistochemical staining.

FIG. 1G is a diagram confirming and quantifying the infiltration of CD4⁺ T cells in a tumor caused by Lsp1 deficiency by immunohistochemical staining.

FIG. 1H is a diagram confirming and quantifying the infiltration of CD8⁺ T cells in a tumor caused by Lsp1 deficiency by immunohistochemical staining.

FIG. 2A is a diagram confirming a ratio of CD3-CD19⁺ B cells and CD3-NK 1.1⁺ NK cells in tumor-infiltrating CD45⁺ leukocytes derived from WT and Lsp1 KO mice.

FIG. 2B is a diagram confirming distribution frequency of T_{reg} cells (Foxp3⁺ regulatory T cells) in tumor-infiltrating CD4⁺ T cells derived from WT and Lsp1 KO mice.

FIG. 2C is a diagram confirming distribution frequency of CD11b⁺ in CD45⁺ leukocytes from WT and Lsp1 KO mice, distribution frequency of TAMs (Ly-6C^{low}F4/80^{high} cells) in CD11b⁺, and distribution frequency of M1-like TAMs (CD206^{low}MHCII^{high} cells) or M2-like TAMs (CD206^{high}MHCII^{low} cells) in Ly-6C^{low}F4/80^{high} cells.

FIG. 3A is a diagram schematically illustrating a method for producing a T cell-specific Lsp1 overexpressed mice.

FIG. 3B is a diagram analyzing the expression level of Lsp1 mRNA in CD4⁺ T cells in the spleen of the T cell-specific Lsp1 overexpressed mice by qRT-PCR.

FIG. 3C is a diagram analyzing the expression level of Lsp1 mRNA in CD8⁺ T cells in the spleen of the T cell-specific Lsp1 overexpressed mice by qRT-PCR.

FIG. 3D is a diagram confirming tumor growth in the T cell-specific Lsp1 overexpressed mice.

FIG. 3E is a diagram confirming tumor volume in the T cell-specific Lsp1 overexpressed mice.

FIG. 3F is a diagram confirming tumor weight in the T cell-specific Lsp1 overexpressed mice.

FIG. 3G is a diagram confirming B16 melanoma-infiltrated T cells in T cell-specific Lsp1 overexpressed mice using flow cytometry.

FIG. 3H is a diagram confirming and quantifying B16 melanoma-infiltrated T cells in T cell-specific Lsp1 overexpressed mice using flow cytometry.

FIG. 4A is a diagram confirming a ratio (%) of CD3-CD19⁺ B cells and CD3-NK 1.1⁺ NK cells in tumor-infiltrating CD45⁺ leukocytes derived from WT and Lsp1 Tg mice.

FIG. 4B is a diagram confirming distribution frequency of T_{reg} cells (Foxp3⁺ regulatory T cells) in tumor-infiltrating CD4⁺ T cells derived from WT and Lsp1 Tg mice.

FIG. 4C is a diagram confirming distribution frequency of CD11b⁺ in CD45⁺ leukocytes derived from WT and Lsp1 Tg mice, distribution frequency of TAMs (Ly-6C^{low}F4/80^{high} cells) in CD11b⁺, and distribution frequency of M1-like TAMs (CD206^{low}MHCII^{high} cells) or M2-like TAMs (CD206^{high}MHCII^{low} cells) in Ly-6C^{low}F4/80^{high} cells.

FIG. 5A is a diagram confirming the expression of LSP1 in T cells and its role in T cell migration through T cell migration analysis performed with spleen CD8⁺ T cells.

FIG. 5B is a diagram confirming the expression of LSP1 in T cells and its role in T cell migration by the expression level of LSP1 in tumor-infiltrating CD4⁺ T cells and CD8⁺ T cells.

FIG. 5C is a diagram confirming LSP1 expression in T cells and its role in T cell migration by increasing LSP1 expression in T cells stimulated with anti-CD3/CD28 Abs and IFN-γ.

FIG. 5D is a diagram confirming LSP1 expression in T cells and its role in T cell migration by increasing LSP1 expression in T cells stimulated with anti-CD3/CD28 Abs and IFN-γ.

FIG. 6A is a Venn diagram showing the overlap between cytotoxicity-related genes and DEGs in Lsp1 KO T cells.

FIG. 6B is a diagram illustrating normalized enrichment scores (NES) of 10 GOBP terms related to a biological process of ‘cytotoxicity’ in Lsp1 KO T cells.

FIG. 6C is a diagram illustrating a GSEA plot of ‘apoptosis’ and ‘leukocyte-mediated cytotoxicity’ enriched in Lsp1 KO T cells stimulated with media or anti-CD3/28 Abs.

FIG. 7A is a diagram quantifying the spleen weights of tumor-bearing WT and Lsp1 KO mice.

FIG. 7B is a diagram quantifying the numbers of splenocytes in tumor-bearing WT and Lsp1 KO mice.

FIG. 7C is a diagram confirming compositions of T cells in the spleens of tumor-bearing WT and Lsp1 KO mice by flow cytometry.

FIG. 7D is a diagram confirming the expression of IL-2, TNF-α, and IFN-γ in CD4⁺ T cells in the spleens of tumor-bearing WT and Lsp1 KO mice by flow cytometry.

FIG. 7E is a diagram confirming the expression of IL-2, TNF-α, and IFN-γ in CD8⁺ T cells in the spleens of tumor-bearing WT and Lsp1 KO mice by flow cytometry.

FIG. 8A is a diagram confirming the expression of TNF-α and IFN-γ in tumor-infiltrating T cells of Lsp1 KO mice.

FIG. 8B is a diagram confirming the expression of TNF-α and IFN-γ in tumor-infiltrating T cells of Lsp1 KO mice.

FIG. 8C is a diagram confirming the expression of TNF-α and IFN-γ in tumor-infiltrating T cells of Lsp1 Tg mice.

FIG. 8D is a diagram confirming the expression of TNF-α and IFN-γ in tumor-infiltrating T cells of Lsp1 Tg mice.

FIG. 9A is a diagram quantifying the spleen weights in WT and Lsp1 Tg mice inoculated with B16 melanoma.

FIG. 9B is a diagram quantifying the numbers of splenocytes in WT and Lsp1 Tg mice inoculated with B16 melanoma.

FIG. 9C is a diagram confirming splenocytes in WT and Lsp1 Tg mice inoculated with B16 melanoma by flow cytometry.

FIG. 9D is a diagram confirming distribution frequencies of IL-2, TNF-α, and IFN-γ in CD4⁺ T cells in the spleens of WT and Lsp1 Tg mice inoculated with B16 melanoma by flow cytometry.

FIG. 9E is a diagram confirming distribution frequencies of IL-2, TNF-α, and IFN-γ in CD8⁺ T cells in the spleens of WT and Lsp1 Tg mice inoculated with B16 melanoma by flow cytometry.

FIG. 10A is a diagram confirming adoptive transfer of Lsp1 KO or Lsp1 Tg T cells to Rag1 KO mice in tumor progression by flow cytometry.

FIG. 10B is a diagram confirming adoptive transfer of Lsp1 KO or Lsp1 Tg T cells to Rag1 KO mice in tumor progression.

FIG. 10C is a diagram schematically illustrating a process for confirming an effect of enhancing an anti-tumor effect of an anti-PD-1 antibody caused by Lsp1 deficiency in tumor progression.

FIG. 10D is a diagram confirming an effect of enhancing an anti-tumor effect of an anti-PD-1 antibody caused by Lsp1 deficiency in tumor progression.

FIG. 10E is a diagram illustrating therapeutic significance of LSP1 deficiency.

### BEST MODE FOR THE INVENTION

Hereinafter, an embodiment of the present invention will be described in detail with reference to the accompanying drawings. However, the following embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

In addition, terminologies used in the present invention are terminologies used to properly express embodiments of the present invention, which may vary according to a user, an operator's intention, or customs in the art to which the present invention pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

In an aspect, the present invention relates to a pharmaceutical composition for preventing or treating cancer including a Leukocyte-specific protein 1 (LSP1) expression inhibitor as an active ingredient.

In one embodiment, the expression inhibitor may be small interfering RNA (siRNA), small hairpin RNA (shRNA), miRNA, antisense oligonucleotide, or nucleic acid aptamer.

In one embodiment, the expression inhibitor may be a T cell-specific LSP1 expression inhibitor.

In one embodiment, the expression inhibitor may include crRNA (CRISPR RNA) including a nuclease-deactivated Cas12a or Cas9 protein, a direct repeat (DR), and a protospacer complementary to the LSP1 gene.

In one embodiment, the cancer may be solid cancer, and the cancer may be any one or more selected from the group consisting of brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, kidney or ureter cancer, renal cell carcinoma, renal pelvic carcinoma, central nervous system tumor, spinal cord tumor, brainstem gliomas and pituitary adenomas.

In one embodiment, the composition of the present invention may increase the number and distribution frequency of T cells.

In one embodiment, the composition of the present invention may increase the infiltration of T cells into tumors.

In one embodiment, the composition of the present invention may regulate the migration of T cells.

In one embodiment, the composition of the present invention may inhibit tumor growth.

In one embodiment, the composition of the present invention may increase the expression and release of TNF-α and IFN-γ in T cells.

In one embodiment, the T cells may be helper T cells or cytotoxic T cells, the helper T cells may be CD4⁺ T cells, and the cytotoxic T cells may be CD8⁺ T cells.

In one embodiment, the composition of the present invention may further include an immune checkpoint inhibitor, and the immune checkpoint inhibitor may be a PD-1 inhibitor or a CTLA-4 inhibitor, and may be an anti-PD-1 antibody.

In one embodiment, the composition of the present invention may be administered simultaneously, separately or sequentially with or from the immune checkpoint inhibitor.

As used in the present invention, the term “expression inhibition” means causing a decrease in expression (to mRNA) or translation (to protein) of a target gene, and preferably means that the expression of the target gene becomes undetectable or exists at an insignificant level thereby.

As used in the present invention, the term “protospacer” refers to a nucleic acid sequence that hybridizes with target DNA.

As used in the present invention, the term “hybridization” refers to a reaction in which one or more nucleic acids react to form a complex, and this complex is stabilized through hydrogen bonds between bases of nucleic acid residues.

As used in the present invention, the term “expression” refers to a process in which a nucleic acid is transcribed from a DNA template (e.g., into mRNA or other RNA transcripts) and/or a process in which transcribed mRNA is subsequently translated into a peptide, polypeptide, or protein.

The composition for inhibiting the gene expression of the present invention may be introduced into cells or organisms in the form of a recombinant vector including DNA encoding a nuclease-deactivated Cas12a protein or Cas9 protein and a recombinant vector including DNA encoding crRNA, introduced into cells or organisms in the form of a mixture including a dCas12a protein or Cas9 protein and crRNA, or a ribonucleic acid protein constituting a complex thereof, or introduced into cells or organisms with a vector including a dCas12a protein or Cas9 protein and crRNA.

In the present invention, the specific sequence of the crRNA may be appropriately selected depending on a type (derived microorganism) of the dCas12a protein or Cas9 protein.

In the present invention, endonuclease such as the dCas12a protein or Cas9 protein may be isolated from microorganisms or non-naturally occur by a recombinant or synthetic method. The endonuclease may further include an element commonly used for intranuclear transfer of cells at an N-terminal or C-terminal (or 5′ end or 3′ end of the nucleic acid molecule encoding the element), but is not limited thereto. The endonuclease protein may be used in the form of a purified protein, or used in the form of DNA encoding the protein, or a recombinant vector including the DNA.

As used in the present invention, the terms “cancer” and “tumor” may be used interchangeably.

In one aspect, the present invention relates to LSP1 gene-deleted T cells.

In one aspect, the present invention relates to a pharmaceutical composition for preventing or treating cancer including LSP1 gene-deleted T cells as an active ingredient.

In one embodiment, the T cells may be activated T cells or CD45⁺ T cells.

In one embodiment, the T cells may be helper T cells or cytotoxic T cells, the helper T cells may be CD4⁺ T cells, and the cytotoxic T cells may be CD8⁺ T cells.

In one embodiment, the T cells may be LSP1-deleted chimeric antigen receptor (CAR)-T cells, and may include a CAR targeting a cancer cell antigen.

In one embodiment, the CAR may include a cancer cell antigen-binding domain, a transmembrane domain, and an intracellular signaling domain.

In one embodiment, the expression and/or function of LSP1 in the T cells may be reduced or eliminated.

In one embodiment, the T cells may be autologous to the subject.

In one embodiment, the composition of the present invention may further include an immune checkpoint inhibitor, and the immune checkpoint inhibitor may be a PD-1 inhibitor or a CTLA-4 inhibitor, and may be an anti-PD-1 antibody.

In one embodiment, the composition of the present invention may be administered simultaneously, separately or sequentially with or from the immune checkpoint inhibitor.

As used in the present invention, the term “prevention” refers to all actions that inhibit or delay the occurrence, development, and recurrence of cancer by administration of the composition according to the present invention.

As used in the present invention, the term “treatment” means all actions that improve or beneficially change the symptoms of cancer and its complications by administration of the composition according to the present invention. Those skilled in the art to which the present invention pertains will be able to determine the degree of improvement, enhancement and treatment by knowing the exact criteria of a disease for which the composition of the present invention is effective by referring to data presented by the Korean Academy of Medical Sciences, etc.

The therapeutically effective dose of the composition of the present invention may vary depending on many factors, for example, an administration method, a target site, a condition of a subject, and the like.

The pharmaceutical composition of the present invention is administered in a pharmaceutically effective dose. As used in the present invention, the term “pharmaceutically effective dose” refers to an amount enough to treat the disease at a reasonable benefit/risk ratio applicable to medical treatment and not cause side effects. An effective dose level may be determined according to factors including the health condition of a subject, the type and severity of cancer, the activity of a drug, the sensitivity to a drug, an administration method, a time of administration, a route of administration, an excretion rate, duration of treatment, and drugs used in combination or simultaneously, and other factors well-known in the medical field. The composition of the present invention may be administered as an individual therapeutic agent or in combination with other therapeutic agents, and may be administered sequentially or simultaneously with existing therapeutic agents, and may be administered singly or multiply. It is important to administer an amount capable of obtaining a maximum effect with a minimal amount without side effects by considering all the factors, which may be easily determined by those skilled in the art.

In one embodiment, the pharmaceutical composition may be one or more formulations selected from the group including oral formulations, external formulations, suppositories, sterile injections and sprays.

The composition of the present invention may include a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using as a suitable method in the art or a method disclosed in Remington's Pharmaceutical Science (Mack Publishing Company, Easton PA, 18th, 1990).

The composition of the present invention may further contain one or more active ingredients exhibiting the same or similar function. The composition of the present invention includes 0.0001 to 10 wt %, preferably 0.001 to 1 wt % of the protein, based on the total weight of the composition.

The pharmaceutical composition of the present invention may further include pharmaceutically acceptable additives. At this time, the pharmaceutically acceptable additive may use starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc, and the like. The pharmaceutically acceptable additive according to the present invention is preferably included in an amount of 0.1 part by weight to 90 parts by weight based on the composition, but is not limited thereto.

The composition of the present invention may be administered parenterally (for example, intravenously, subcutaneously, intraperitoneally, or topically) or orally depending on a desired method, and most preferably orally administered. The range of the dose thereof varies according to the weight, age, sex, health condition, diet, administration time, administration method, and excretion rate of a subject, the severity of a disease, etc.

Liquid formulations for oral administration of the composition of the present invention correspond to suspensions, internal solutions, emulsions, syrups, etc., and may include various excipients, such as wetting agents, sweeteners, fragrances, and preservatives, in addition to water and liquid paraffin, which are commonly used simple diluents. Formulations for parenteral administration include sterilized aqueous solutions, non-aqueous solvents, suspensions, emulsions, lyophilized agents, suppositories, and the like.

In an aspect, the present invention relates to an anticancer adjuvant for enhancing the activity of an anticancer agent including an LSP1 expression inhibitor or LSP1 gene-deleted T cells as an active ingredient.

In one embodiment, the anticancer agent may be eribulin, carboplatin, cisplatin, Halaven, 5-fluorouracil (5-FU), gleevec, Vincristine, Vinblastine, Vinorelvine, Paclitaxel, Docetaxel, Etoposide, Topotecan, Irinotecan, Dactinomycin, Doxorubicin, Daunorubicin, valrubicin, flutamide, gemcitabine, Mitomycin, or Bleomycin.

In one embodiment, the composition of the present invention may further include an immune checkpoint inhibitor, and the immune checkpoint inhibitor may be a PD-1 inhibitor or a CTLA-4 inhibitor, and may be an anti-PD-1 antibody.

In one aspect, the present invention relates to a method of providing information for diagnosing cancer or predicting prognosis of cancer, including confirming the expression level of LSP1 in T cells isolated from a subject.

As used in the present invention, the term “diagnosis” refers to determining the susceptibility of a subject to a specific disease or disorder, determining whether a subject currently has a specific disease or disorder, determining the prognosis of a subject suffering from a specific disease or disorder (e.g., identifying a pre-metastatic or metastatic cancer condition, determining a stage of cancer, or determining the responsiveness of cancer to be treated), or therametrics (e.g., monitoring the condition of a subject to provide information about therapeutic efficacy).

In an aspect, the present invention relates to a method for predicting the prognosis for anticancer therapy including: 1) measuring the expression level of LSP1 in T cells isolated from a subject; b) performing anticancer therapy; c) re-measuring the expression level of LSP1 in the T cells isolated from the subject; and d) comparing a measured value of step a) with a measured value of step c).

In one embodiment, the expression level may be measured by confirming the expression level of mRNA or the level of a protein encoded by a gene. The amount of mRNA may be confirmed using primer pairs or probes, and an analysis method thereof includes RT-PCR, competitive RT-PCR, real-time RT-PCR, RNase protection assay (RPA), Northern blotting, DNA chips, etc., but is not limited thereto. The amount of protein encoded by the gene may be confirmed using an antibody that specifically binds to the protein. An analysis method thereof includes Western blot, enzyme linked immunosorbent assay (ELISA), radioimmunoassay (RIA), radioimmunodiffusion, Ouchterlony immunodiffusion method, rocket immunoelectrophoresis, tissue immunostaining, immunoprecipitation assay, complement fixation assay, FACS, protein chips, etc., but is not limited thereto.

Further, the present invention provides a pharmaceutical composition for preventing or treating cancer caused by LSP1 overexpression including an LSP1 inhibitor as an active ingredient.

Further, the present invention provides a pharmaceutical composition for preventing or treating cancer caused by LSP1 overexpression including LSP1 gene-deleted T cells as an active ingredient.

Further, the present invention provides a method for preventing or treating cancer including administering a pharmaceutical composition including a Leukocyte-specific protein 1 (LSP1) expression inhibitor as an active ingredient to a subject.

As used in the present invention, the term “subject” refers to all animals, including humans, that have already developed or may develop cancer. The composition including the expression inhibitor or cells of the present invention may be administered to a subject, thereby efficiently preventing and treating the disease. For example, the composition of the present invention may be used to treat humans suffering from various cancers or metastatic cancer. In addition, the composition of the present invention may be used to treat mice or pigs suffering from various carcinomas or tumors. The composition of the present invention may be administered in combination with existing anticancer drugs or anticancer adjuvants.

The composition of the present invention is administered in a pharmaceutically effective dose. The term “pharmaceutically effective dose” refers to an amount enough to treat diseases at a reasonable benefit/risk ratio applicable to medical treatment, and an effective dose level may be determined according to factors including a type of subject, the severity, age, gender, a type of infected virus, the activity of a drug, sensitivity to a drug, a time of administration, a route of administration, an excretion rate, duration of treatment, and drugs to be simultaneously used, and other factors well-known in the medical field. The composition of the present invention may be administered as an individual therapeutic agent or administered in combination with other therapeutic agents, and sequentially or simultaneously administered with conventional therapeutic agents. In addition, the pharmaceutical composition may be administered singly or in multiple. It is important to administer an amount capable of obtaining a maximum effect with a minimal amount without side effects in consideration with all the factors, and the amount thereof may be easily determined by those skilled in the art.

Further, the present invention provides a method for preventing or treating cancer including administering a pharmaceutical composition including LSP1 gene-deleted T cells as an active ingredient to a subject.

Further, the present invention provides a method for preventing or treating cancer caused by LSP1 overexpression including administering a pharmaceutical composition including an LSP1 inhibitor as an active ingredient to a subject.

Further, the present invention provides a method for preventing or treating cancer caused by LSP1 overexpression including administering a pharmaceutical composition including LSP1 gene-deleted T cells as an active ingredient to a subject.

Hereinafter, the present invention will be described in more detail with reference to the following Examples. However, the following Examples are only intended to embody the contents of the present invention, and the present invention is not limited thereto.

### MODES FOR THE INVENTION

### Example 1. Tumor Growth Inhibition by LSP1 Inhibition

### 1-1. Tumor Growth Inhibition Effect by LSP1 Inhibition

5×10⁵ B16 melanomas were re-suspended in PBS, and then injected subcutaneously into the right flanks of 8-12-week-old WT mice and Lsp1 KO mice (Dr. Laurent Sabbagh, University of Montreal, Montreal, Quebec, Canada), and tumor volume was measured with a caliper every 2 to 3 days and calculated using Equation 1 below to confirm the tumor growth for 3 weeks.

$$V ( mm 3 ) = D \times d 2 \times 0.52 \qquad [ Equation 1 ]$$

D (mm): Vertical diameter of largest tumor; and
d (mm): Vertical diameter of smallest tumor

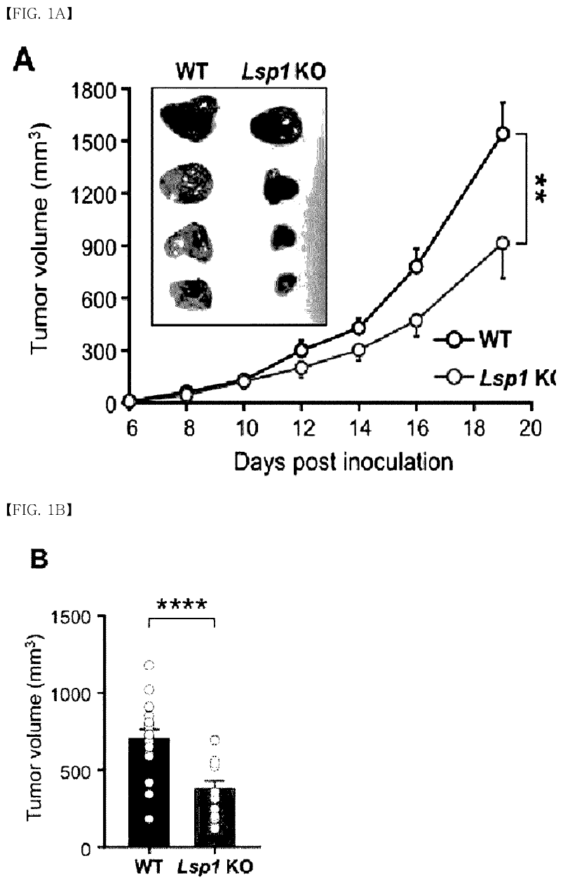

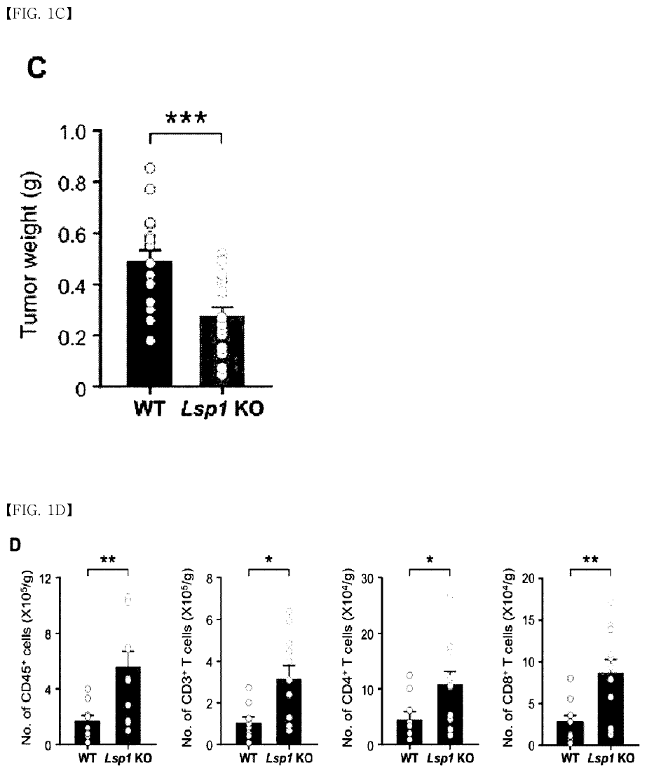

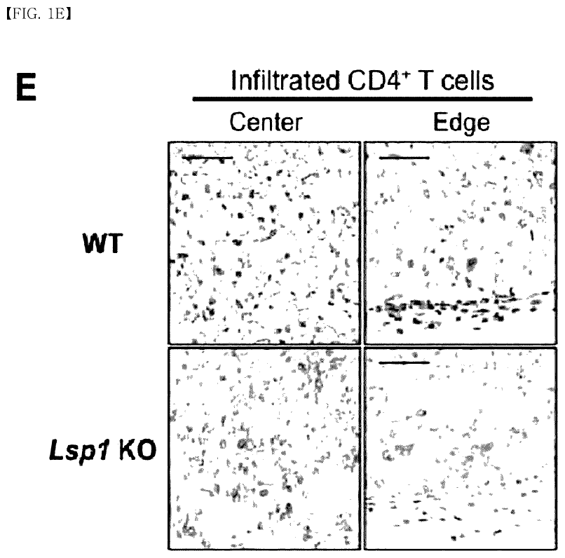

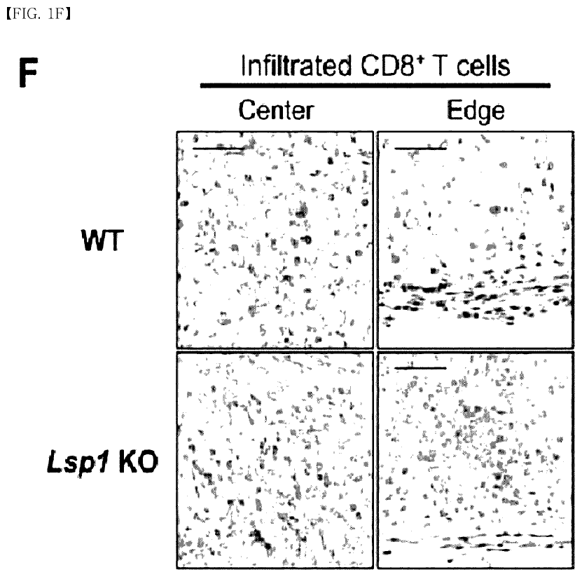

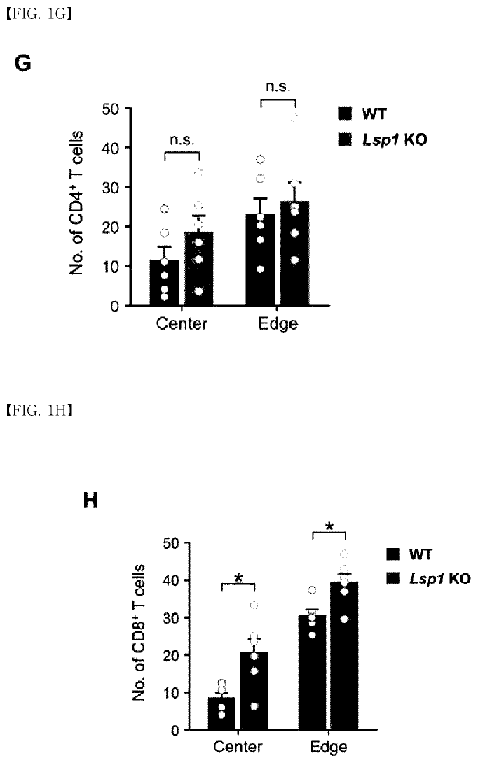

As a result, the tumor growth was significantly reduced in Lsp1 KO mice compared to WT mice (FIG. 1A). In addition, after 14 days of tumor inoculation, the tumor volume and weight in Lsp1 KO mice were significantly lower than those in WT mice (FIGS. 1B and 1C).

### 1-2. Tumor Infiltration Promotion Effect of T Cells by LSP1 Inhibition

In order to confirm an immune contexture in tumor microenvironments (TME) affected by Lsp1 deficiency in mice with the melanoma above, tumor-infiltrating leukocytes (TILs) from tumor-bearing WT and Lsp1 KO mice were analyzed by flow cytometry when the average tumor volume reached about 700 mm³ in WT mice. Specifically, in order to isolate TILs, WT and Lsp1 KO mice were sacrificed when the average tumor volume of WT mice reached 500 or 700 mm³, and primary tumors were mechanically excised and isolated. Thereafter, a tumor cell suspension was obtained by filtration through a 70 μm cell filter, and TILs were isolated using a Ficoll-Hypaque (GE Healthcare, Chicago, IL) density gradient centrifuge. The isolated single-cell suspension was washed with PBS and then flow cytometry was performed. For flow cytometry, surface staining was performed for 30 minutes at 4° C. with fluorochrome-labeled anti-mouse antibodies such as CD45 (30-F11, BD Pharmingen, Franklin Lakes, NJ), CD3 (145-2C11, Invitrogen, Carlsbad, CA), CD4 (GK1.5, Biolegend, San Diego, CA), and CD8 (53-6.7, Biolegend). Thereafter, the cells were resuspended in FACS buffer and flow cytometry was performed using FACS CantoII (BD Biosciences) or LSR Fortessa (BD Biosciences) with DIVA software, and as a result, the obtained data were analyzed using FlowJo software (FlowJo, LLC, Franklin Lakes, NJ). As the result, it was showed that the tumor from Lsp1 KO mice had significantly higher numbers of infiltrated CD45⁺ cells, CD3⁺, CD4⁺, and CD8⁺ T cells than the tumor from WT mice (FIG. 1D). From this, it may be inferred that the reduced growth of melanoma in Lsp1 KO mice may be related to increased cell number rather than T cell composition.

In addition, immunohistochemical staining of CD8⁺ T cells and CD4⁺ T cells was performed to confirm T cell infiltration in the tumor. Specifically, 7 μm sections of OCT-embedded tumor tissue were fixed with cold acetone for 10 minutes at −20° C., and the sections were incubated in 0.3% H₂O₂ for 30 minutes at room temperature to quench endogenous peroxidase, and then the tissue was blocked with 10% normal donkey serum at room temperature for 1 hour. Thereafter, the tissue sections were incubated with rat anti-mouse CD4 (1:1000, Biolegend) or rat anti-mouse CD8 (1:1000, Biolegend) Ab overnight at 4° C., and each slide was washed three times with PBS, and the detection was performed with a VECTASTAIN Elite ABC HRP kit (Vector Laboratories) using anti-rat secondary Ab (1:100, Vector Laboratories, Burlingame, CA). Positive cells were detected using 3,3′-diaminobenzidine tetrahydrochloride (DAB; Vector Laboratories) and counterstained with hematoxylin. Images were obtained with a Pannoramic MIDI slide scanner (3DHISTECH), and the stained positive cells were manually counted in six fields on two different slides for each mouse. As a result, unlike CD4⁺ T cells, the infiltration of CD8⁺ T cells was significantly higher in both the center and edge of the tumor in Lsp1-deficient mice than in WT mice (FIGS. 1E to 1H), and thus it was confirmed that Lsp1 deficiency promoted the infiltration of CD8⁺ T cells into the tumor center.

### 1-3. Identification of Immune Cell Groups

In order to confirm the effect in tumors by LSP1 inhibition on other immune cell groups other than T cells constituting TME, the distribution frequencies and numbers of NK1.1⁺ NK cells, CD19⁺ B cells, regulatory T cells (T_{reg} cells: Foxp3⁺ CD4⁺ T cells), and tumor-associated macrophages (TAMs) in the tumor were confirmed in tumors of Lsp1 KO mice and WT mice by flow cytometry.

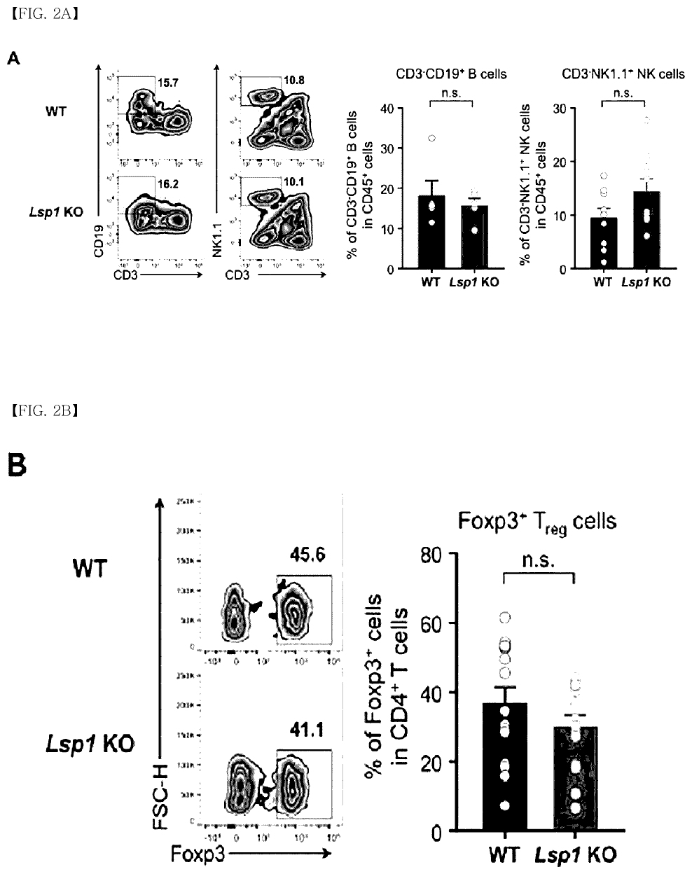

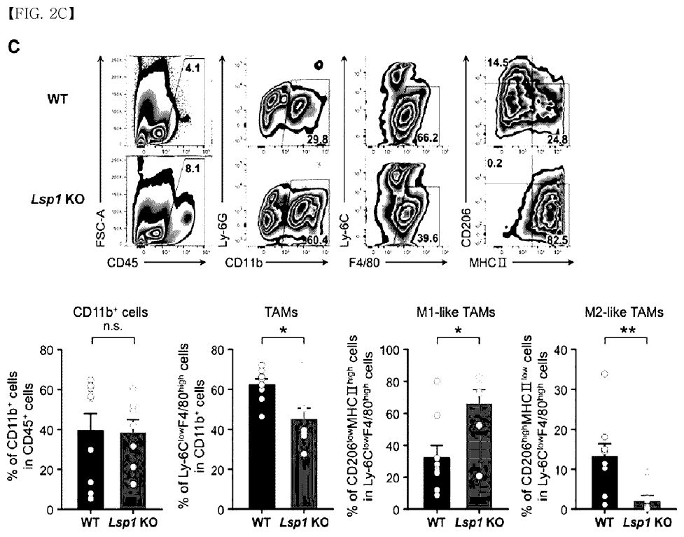

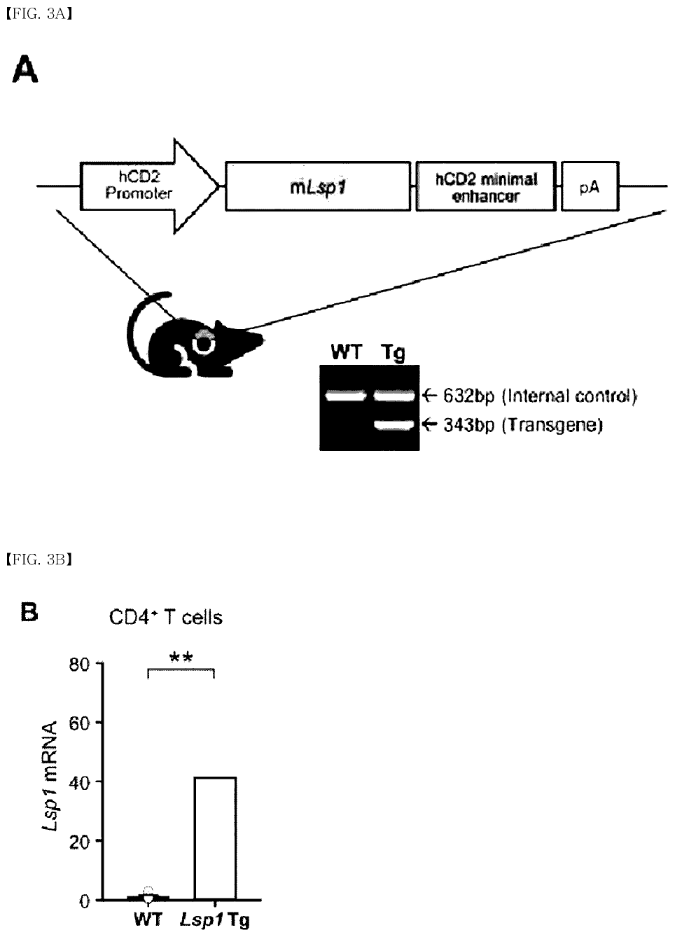

As a result, although the absolute number of TILs was significantly high in the tumor of Lsp1 KO mice, there was no difference in the distribution frequency of NK1.1⁺ NK cells and CD19⁺ B cells in the tumor (FIG. 2A), and there was no difference in the distribution frequency of regulatory T cells (T_{reg} cells: Foxp3⁺ CD4⁺T cells) (FIG. 2B). In addition, while CD11b⁺ myeloid cells appeared similarly in Lsp1 KO and WT mice, the distribution frequency of CD11b⁺Ly6C^{low}F4/80^{high} tumor-associated macrophages (TAMs) was significantly reduced in the tumor of Lsp1 KO mice compared to WT mice (FIG. 2C). Through the results, it was noticeable that among TAMs in the tumor of Lsp1 KO mice, pro-inflammatory M1-like (CD206^{low} MHCII^{high}) TAMs were significantly increased and anti-inflammatory M2-like (CD206^{high} MHCII^{low}) TAMs were significantly decreased (FIG. 2C).

Through the results, it may be seen that Lsp1-deficient mice enhance the infiltration of CD8⁺ T cells and the infiltration of pro-inflammatory M1-like TAMs compared to M2-like TAMs to create an anti-tumor immune environment.

### Example 2. Tumor Growth Promotion by LSP1 Overexpression

### 2-1. Tumor Growth Promotion Effect by LSP1 Overexpression

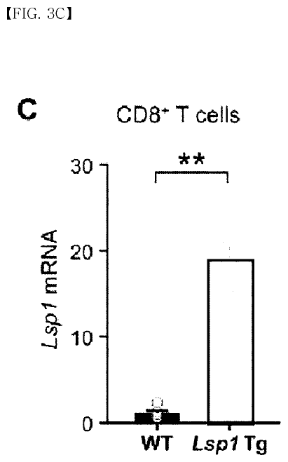

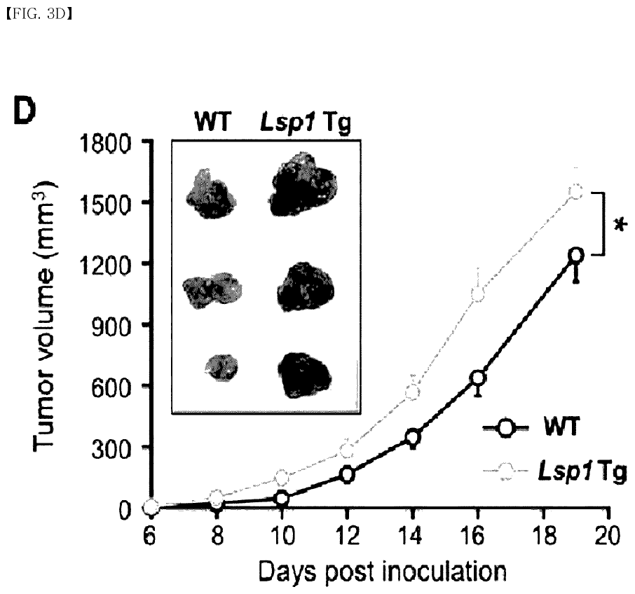

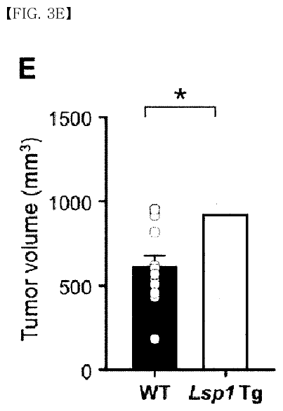

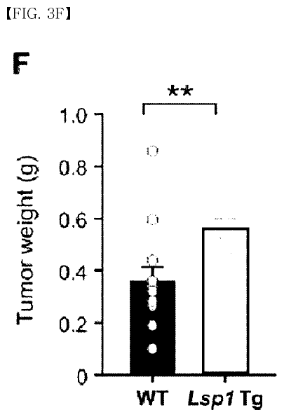

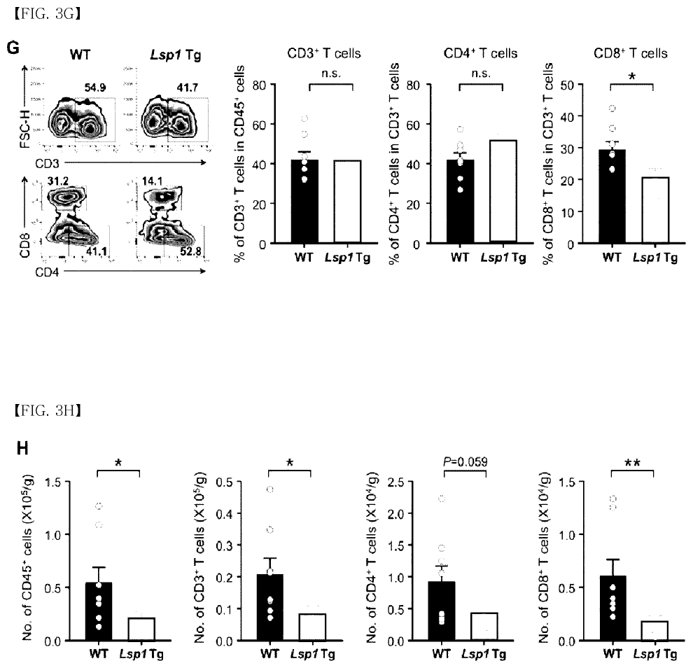

To confirm the T cell-specific effect of LSP1 during tumor development, transgenic (Tg) mice overexpressing Lsp1 in T cells were prepared (FIG. 3A). Specifically, cDNA of mouse Lsp1 was cloned into a leukocyte-specific expression cassette containing a human CD2 promoter, a construct was directly injected into the prokaryote of a fertilized egg, and genomic DNA was analyzed by PCR to select a transgenic founder. Thereafter, total RNA was isolated from CD4⁺ and CD8⁺ T cells in the spleen using an RNeasy Mini kit (Qiagen, Hilden, Germany) and converted to cDNA using RevertAid Reverse Transcriptase (Thermo Fisher Scientific). Thereafter, the expression level of Lsp1 mRNA was analyzed by a CFX96 real-time PCR system using SYBR Green PCR premix (Bio-Rad, Hercules, CA) and primers (5′-CCAGCCCTTTGGCCTTAGAA-3′ and 5′-TGGAAATGGGCAAGGTTGGT-3′) and compared with WT mice to verify the Lsp1 Tg mice. The Lsp1 Tg mice and WT mice prepared above were subcutaneously inoculated with melanoma as in Example 1 above, and the growth degree of the tumor was confirmed. As a result, in contrast to Lsp1 KO mice, Lsp1 Tg mice (FIGS. 3B and 3C), in which CD4⁺ and CD8⁺ T cells expressed significantly more Lsp1 mRNA compared to WT mice, showed significantly accelerated tumor growth compared to WT mice for 3 weeks (FIG. 3D). The tumor volume and weight of Lsp1 Tg mice were also significantly higher than those of WT mice at day 14 after tumor inoculation (FIGS. 3E and 3F).

### 2-2. Identification of Immune Cell Groups

In order to evaluate immune cell groups in the TME of Lsp1 Tg mice, when the tumor volume of WT mice reached about 500 mm³, the tumor was collected and the number and distribution frequency of TILs were analyzed by flow cytometry.

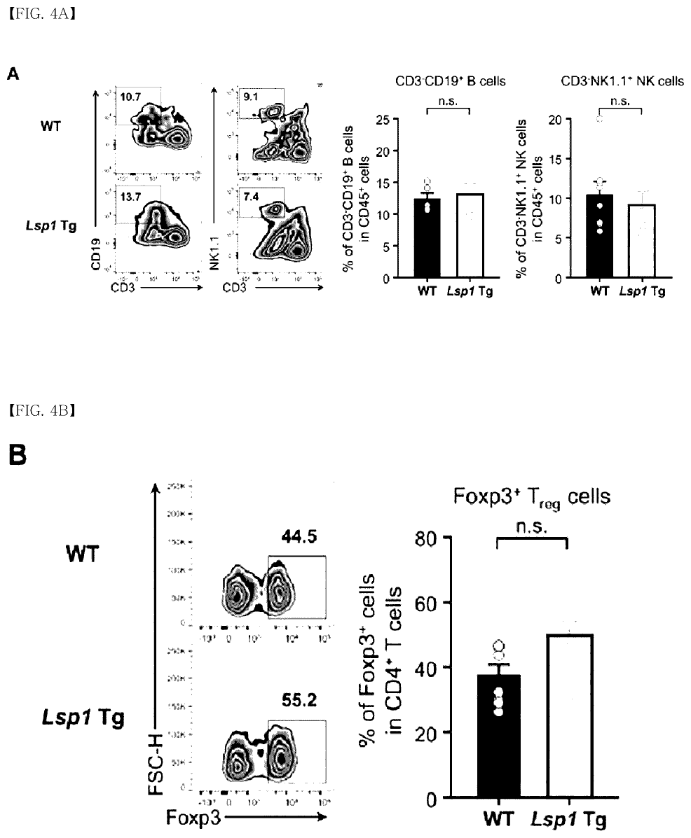

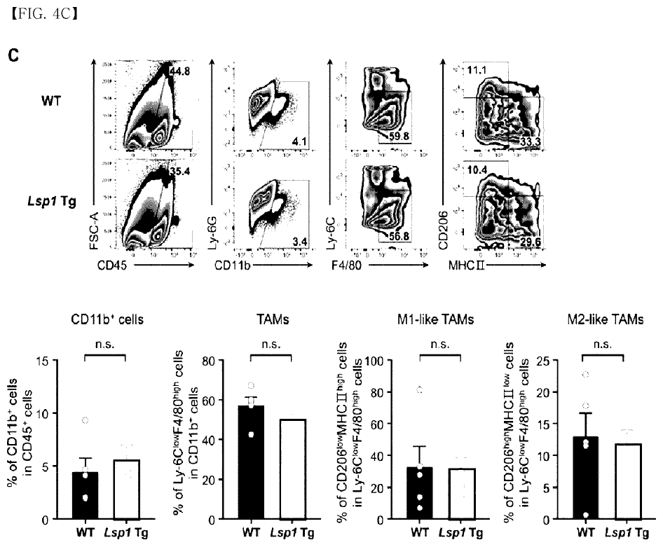

As a result, it was shown that the distribution frequencies of tumor-infiltrating CD3⁺ T cells and CD4⁺ T cells were similar between WT and Lsp1 Tg mice, but in contrast to the results in Lsp1 KO mice, it was shown that the distribution frequency of CD8⁺ T cells was reduced in Lsp1 Tg mice compared to WT mice (FIG. 3G). In addition, the absolute numbers of CD45⁺ T cells, CD3⁺ T cells, and especially CD8⁺ T cells in the tumor were significantly lower in Lsp1 Tg mice than in WT mice (FIG. 3H). In addition, since Lsp1 overexpression was specific for T cells, there was no difference in distribution frequency of NK1.1⁺ NK cells, CD19⁺ B cells, and CD11b⁺ myeloid cells in the tumor (FIGS. 4A to 4C).

Through the results, it may be seen that specific overexpression of Lsp1 in T cells enhances the growth of B16 melanoma, which is associated with a decrease in the number and distribution frequency of TILs, especially CD8⁺ T cells.

### Example 3. Regulation of T Cell Migration by LSP1

### 3-1. Inhibition of CD8⁺ T Cell Migration by LSP1

In Example above, since genetic Lsp1 inhibition (deficiency) regulated T cell infiltration in B16 melanoma, to confirm whether LSP1 directly regulated T cell migration, it was confirmed whether LSP1 regulated CD8⁺ T cell migration in response to CXCL9 and CXCL10, which trafficked CD8⁺ T cells to the tumor microenvironment and promoting interactions between tumor-specific T cells and dendritic cells in the TME during anti-PD-1 therapy. Specifically, in order to confirm the chemotaxis of WT and Lsp1 KO CD8⁺ T cells, murine CXCL9 and CXCL10 (R&D Systems, Minneapolis, MN) were diluted and located in a transfer medium (RPMI1640 with 0.1% FBS) in a lower chamber of a 24-well plate equipped with a Transwell insert (Corning Inc., Corning, NY) with a pore size of 5 μm and 500,000 CD8⁺ T cells were dispensed into a migration medium in an upper chamber. After 4 hours of incubation at 37° C., the cells migrated to the lower chamber were counted with a hemocytometer.

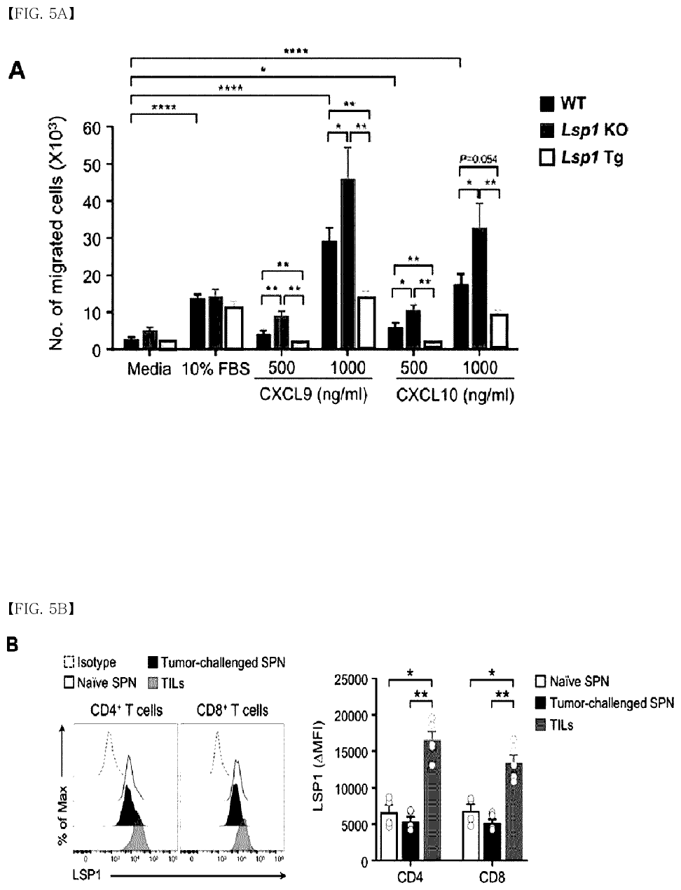

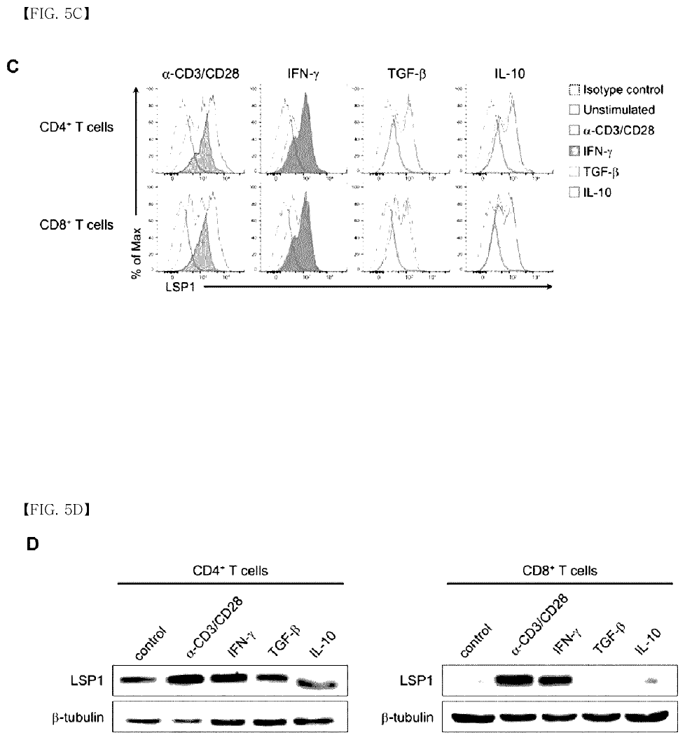

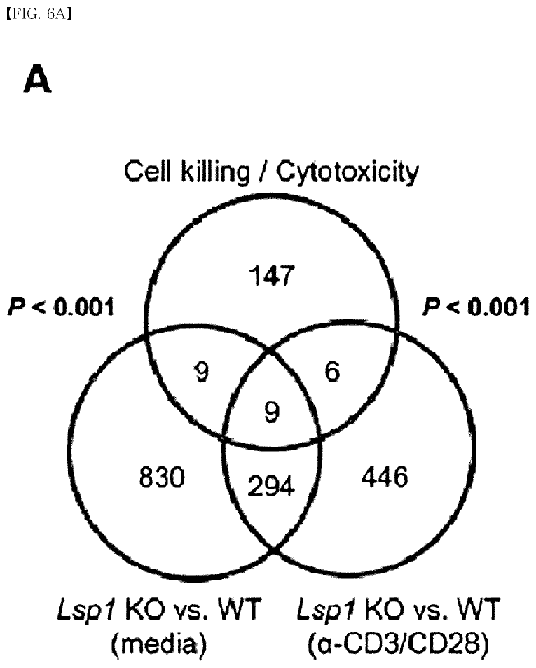

As a result, it was confirmed that Lsp1-deficient CD8⁺ T cells showed a more significant chemotactic response to CXCL9 and CXCL10 than WT CD8⁺ T cells (FIG. 5A). On the other hand, Lsp1-overexpressed CD8⁺ T cells showed reduced chemotactic migration compared to WT and Lsp1-deficient CD8⁺ T cells (FIG. 5A), and thus, it was confirmed that LSP1 negatively regulated the migration of CD8⁺ T cells. However, CD8⁺ T cell migration stimulated with 10% FBS had no difference among three types of CD8⁺ T cells (FIG. 5A), and thus, it was suggested that the regulation of CD8⁺ T cell migration of LSP1 was specific to CXCL9 and CXCL10.

### 3-2. Confirmation of Pathological Relation of TIL and LSP1

In order to confirm a pathological relation between T cells and LSP1 under tumor conditions, the expression level of LSP1 in TILs of B16 melanoma was confirmed by flow cytometry. Specifically, cells were isolated from the spleens of normal mice or tumor-bearing mice and prepared as a single-cell suspension. CD4⁺ T cells or CD8⁺ T cells were purified by magnetic separation using anti-CD4 beads or anti-CD8 beads (Miltenyi Biotec, Bergisch Gladbach, Germany). To detect LSP1 expression in T cells, the purified CD4⁺ T cells or CD8⁺ T cells were stimulated in complete media with recombinant IFN-γ (10 ng/ml, R&D Systems), transforming growth factor beta (TGF-β) (2 ng/ml, R&D Systems), interleukin 10 (IL-10) (10 ng/ml, R&D Systems), or anti-mouse CD28 Ab (1 μg/ml, 37.51, Invitrogen) together with anti-mouse CD3 Ab (1 μg/ml, 145-2C11, Invitrogen) for 72 hours. The cultured cells were collected and stained, and intracellular LSP1 expression was detected by flow cytometry. In addition, the T cells were lyzed with a lysis buffer, electrophoresed on a 12% SDS-PAGE gel to separate proteins, transferred to a PVDF membrane, and electroblotted. The membranes were incubated with LSP1 (1:1000, Cell Signaling Technology) antibody or β-tubulin (1:1000, Abcam, Cambridge, U.K.) antibody, and bands were visualized using an enhanced chemiluminescent detection system (Thermo fisher scientific).

As a result of confirming the LSP1 expression level, CD4⁺ T cells infiltrated into B16 melanoma showed a significantly higher expression level of LSP1 than splenic T cells from tumor-free mice (FIG. 5B). In addition, CD8⁺ T cells infiltrated into B16 melanoma showed a similar result, and thus, it could be seen that high expression of LSP1 in T cells may be induced by B16 melanoma. To confirm how upregulation of LSP1 expression occurred in in vivo tumor-infiltrating T cells, as a result of confirming through flow cytometry what type of tumor-related stimulus may induce LSP1 expression, when T cells were stimulated with anti-CD3/CD28 Abs or the pro-inflammatory cytokine, IFN-γ, LSP1 expression was strongly increased on both CD4⁺ and CD8⁺ T cells, whereas TGF-β and the anti-inflammatory cytokine, IL-10 failed to upregulate the LSP1 expression (FIG. 5C). In addition, the T cell receptor activation and increased LSP1 expression by IFN-γ were also confirmed by Western blot assay (FIG. 5D).

Considering that it was confirmed through Example that LSP1 negatively regulated the migration of T cells, these results suggest that B16 melanoma may evade the anti-tumor activity of host T cells by upregulating the LSP1 expression on T cells in the TME.

### Example 4. Increased Cytotoxicity of Lsp1-Deficient T Cells

### 4-1. Confirmation of Increased T Cell Cytotoxicity by Lsp1 Deficiency

To confirm whether the regulation of LSP1 in tumor growth was completely caused by the regulation of T cell migration, the effect of LSP1 on cytotoxicity of T cells, which was an essential step for tumor-infiltrating T cells to inhibit tumor growth, was confirmed. To this end, a gene expression profile of Lsp1 KO T cells (GSE75123) was analyzed compared to WT T cells focusing on T cell-mediated cytotoxicity. Specifically, total RNA was isolated from splenic T cells of Lsp1 KO and WT mice stimulated with anti-CD3/CD28 Abs for 6 hours, reversely transcribed, and amplified, and then hybridized on an array chip (SurePrint G3 Mouse GE 8×60K Microarray, Agilent) containing 62,976 probes for 24,241 annotated genes (GSE75123). After normalization, the log 2 fold change value and P value of each gene were calculated. The cutoff value of DEGs in Lsp1 KO T cells was |fold change values|>0.58 and P values<0.05. In addition, Gene Set Enrichment Analysis (GSEA) was performed with a clusterProfiler (R package, ver.3.4.6), and the GSEA diagram was generated with enrichrplot (R package). In addition, the significance of overlap between DEGs and cytotoxicity-related genes in Lsp1 KO T cells was determined using a permutation test strategy. A total of 100,000 randomly permuted samples were used to calculate the empirical P-value of overlapping DEGs.

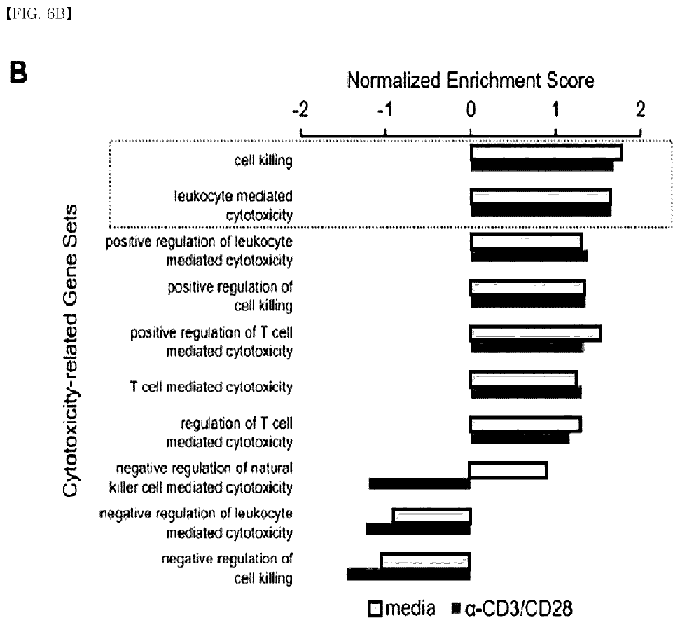

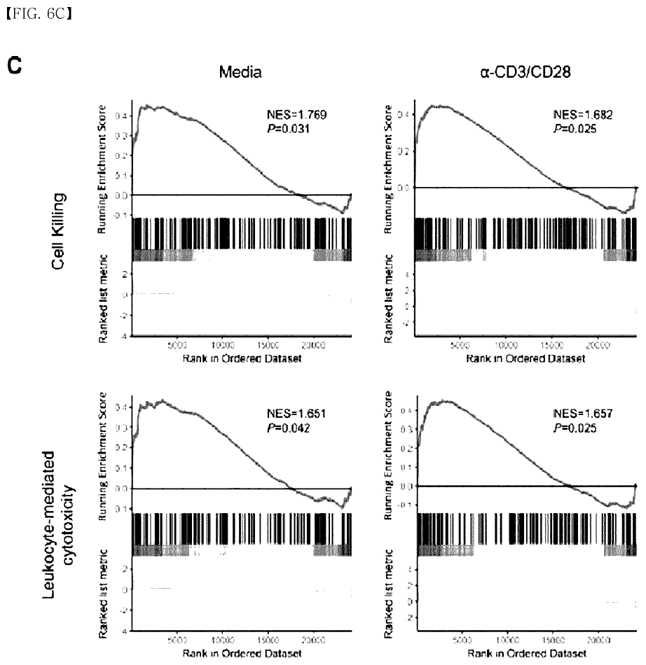

As a result, it was found that among 171 genes representing 10 Gene Ontology Biological Process (GOBP) terms related to cytotoxicity and apoptosis, 24 genes (14%) were significantly overlapped with differentially expressed genes (DEGs) in Lsp1 KO T cells (FIG. 6A). In addition, even in the Gene Set Enrichment Analysis (GSEA) results, it was confirmed that the biological processes of apoptosis and leukocyte-mediated cytotoxicity were increased in Lsp1 KO T cells (FIGS. 6B and 6C), whereas negative regulation of apoptosis was decreased (FIG. 6B). Through this, it may be seen that LSP1 deficiency in T cells is associated with increased tumor-killing cytotoxicity of T cells.

### 4-2. Confirmation of T Cell Cytotoxic Effector Function Promotion by Lsp1 Deficiency

In order to confirm whether cytotoxic effector functions of T cells were promoted by Lsp1 deficiency, the expression levels of representative anti-tumor effector cytokines, IFN-γ and TNF-α, were measured in the splenic and tumor-infiltrating T cells of WT and Lsp1 KO mice by flow cytometry.

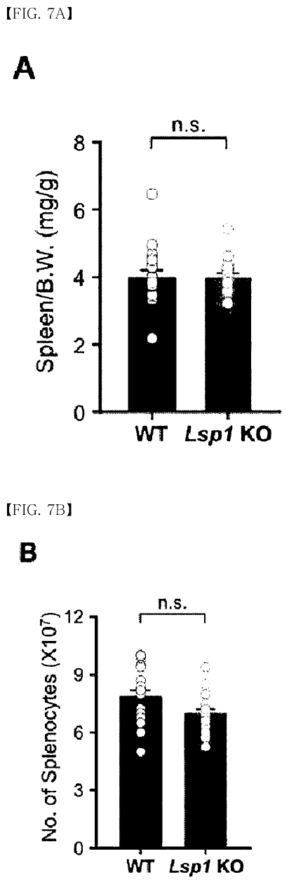

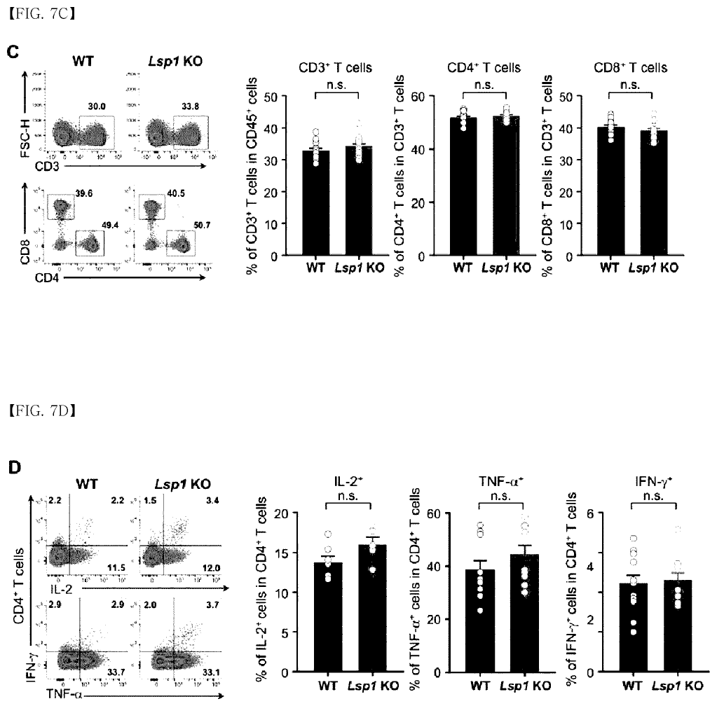

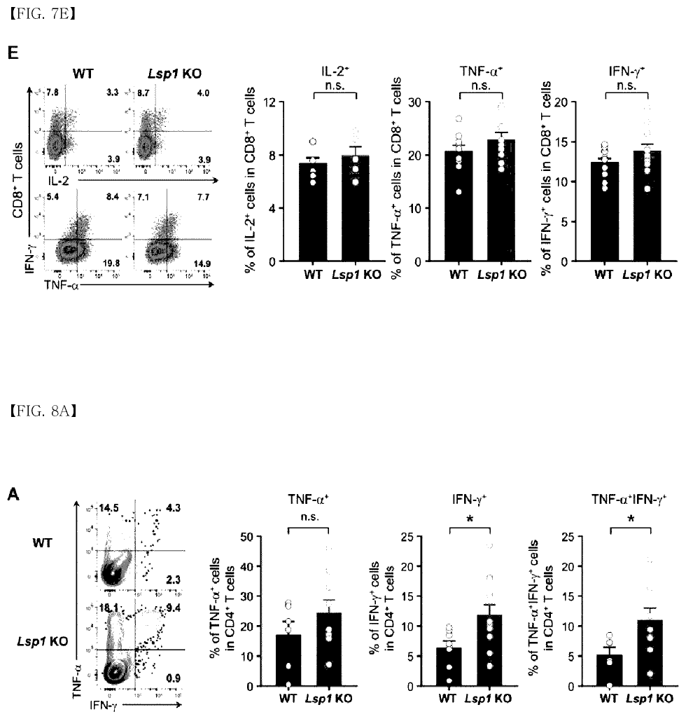

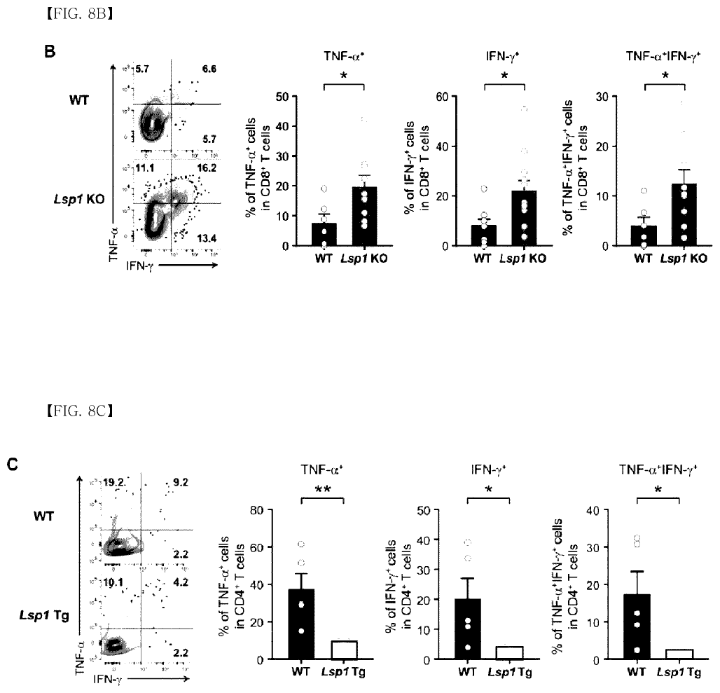

As a result, there were no differences in spleen size, spleen cell number, and splenic CD4⁺ T cells and CD8⁺ T cells between tumor-bearing WT and Lsp1 KO mice (FIGS. 7A, 7B, and 7C). In the spleens of both groups, the distribution frequencies of TNF-α⁺ and IFN-γ⁺ cells among CD4⁺ and CD8⁺ T cells were similar (FIGS. 7D and 7E). On the other hand, in tumors, the distribution frequency of TNF-α⁺ and IFN-γ⁺ cells was significantly higher in infiltrated CD4⁺ and CD8⁺ T cells in Lsp1 KO mice than WT mice (FIGS. 8A to 8D), and thus, it could be seen that Lsp1 deficiency increased anti-tumor immunity by inducing TNF-α⁺ and IFN-η⁺ expression in tumor-infiltrating T cells.

### 4-3. Confirmation of T Cell Cytotoxic Effector Function Reduction by Lsp1 Overexpression

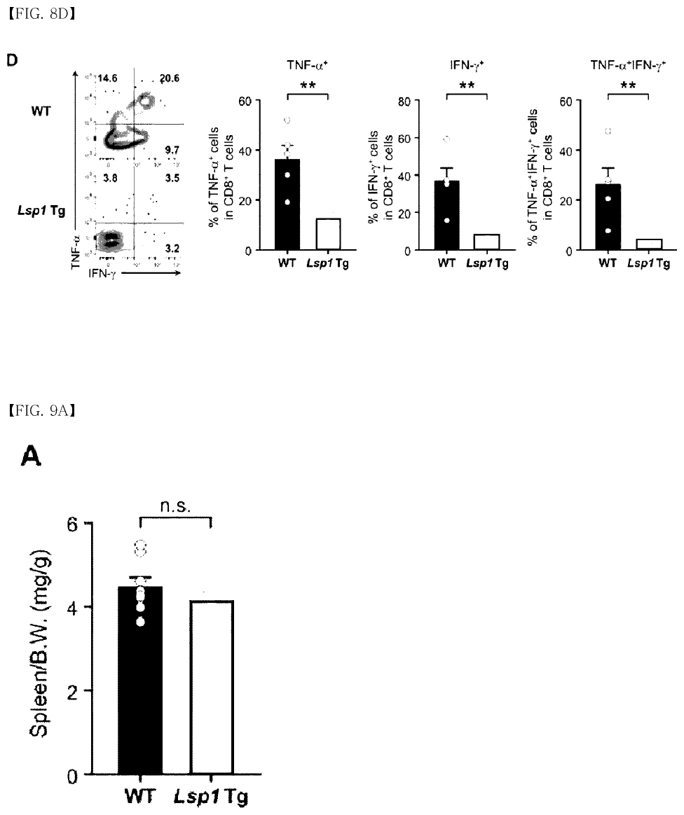

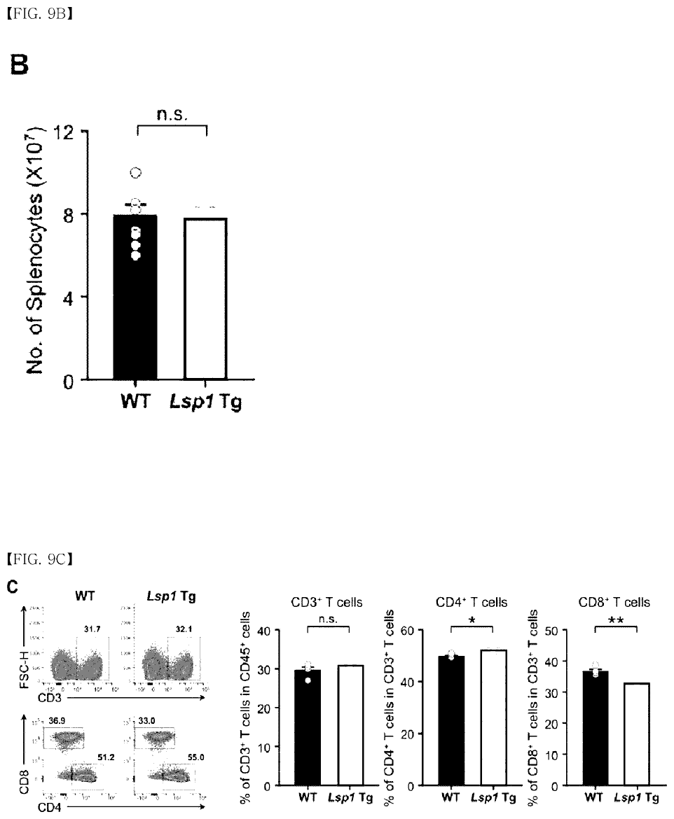

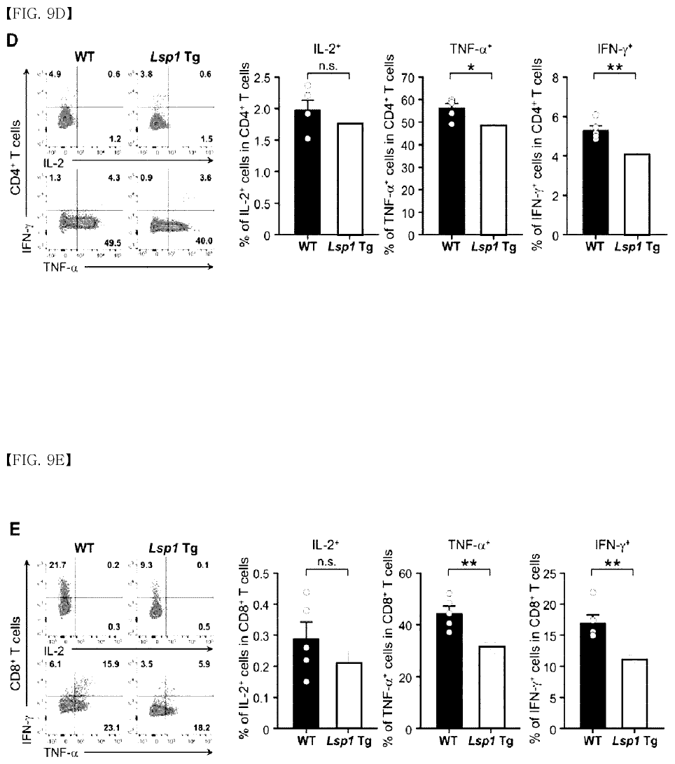

When confirming the spleen weights and spleen cell numbers of Lsp1 Tg mice and WT mice, it was confirmed that the spleen weight and spleen cell number of Lsp1 Tg mice were decreased compared to those of WT mice (FIGS. 9A and 9B).

In addition, on contrary to Example above, as a result of confirming the expression levels of IFN-γ and TNF-α in the splenic and tumor-infiltrating T cells of Lsp1 Tg mice and WT mice by flow cytometry, after tumor inoculation, the distribution frequency of TNF-α and/or IFN-γ+ cells in splenic CD4⁺ and CD8⁺ cells was significantly lower in Lsp1 Tg mice than in WT mice (FIGS. 9C, 9D, and 9E). In addition, Lsp1-overexpressed CD4⁺ and CD8⁺ T cells in tumors showed significantly reduced distribution frequencies of TNF-α⁺ and/or IFN-γ⁺ compared to WT CD4⁺ and CD8⁺ T cells, respectively (FIGS. 9C, 9D, and 9E). While splenic CD8⁺ T cells from Lsp1 Tg mice were less expanded than WT mice, splenic CD4⁺ T cells from Lsp1 Tg mice were more expanded than WT mice (FIG. 9C). Through these results, it may be seen that when Lsp1 is overexpressed in T cells, infiltrated CD8⁺ T cells are significantly reduced and the growth of melanoma is promoted by downregulating TNF-α⁺ and IFN-γ⁺ production by CD8⁺ T cells.

### Example 5. Anti-Tumor Activity Enhancing Effect of Anti-PD-1 Antibody by Lsp1 Deficiency

### 5-1. Anti-Tumor Mediating Effect of Lsp1-Deficient T Cells

Based on the results, assuming that Lsp1-deficient T cells more effectively inhibited tumor growth by increased T cell trafficking and cytotoxic capacity, it was confirmed by performing an adoptive transfer experiment using Lsp1 KO and Lsp1 Tg T cells in Rag1 KO mice deficient in mature T cells and B cells. Specifically, 1×10⁵ B16 melanomas were subcutaneously inoculated into the right flank of Rag1 KO mice, and one day later, T cells were isolated from the spleens of tumor-free Lsp1 KO mice and Lsp1 Tg mice by magnetic separation using a Pan T cell isolation kit (Miltenyi Biotec). PBS as a vehicle or 1×10⁷ T cells derived from Lsp1 KO mice or Lsp1 Tg mice were injected intravenously into the B16 melanoma-bearing Rag1 KO mice. Tumor growth was recorded every 2 days.

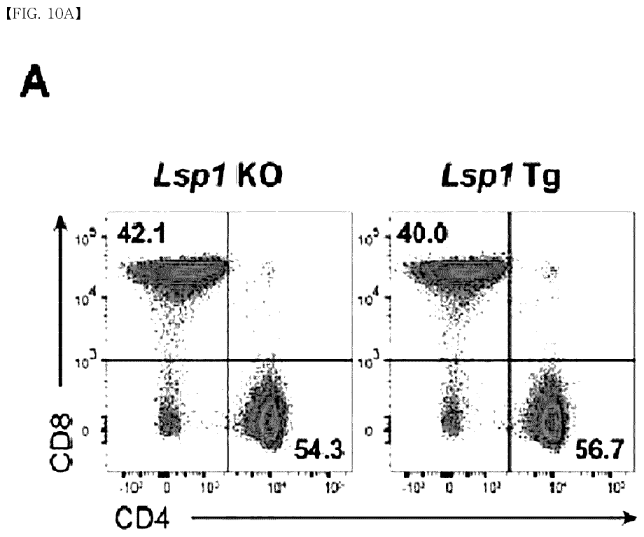

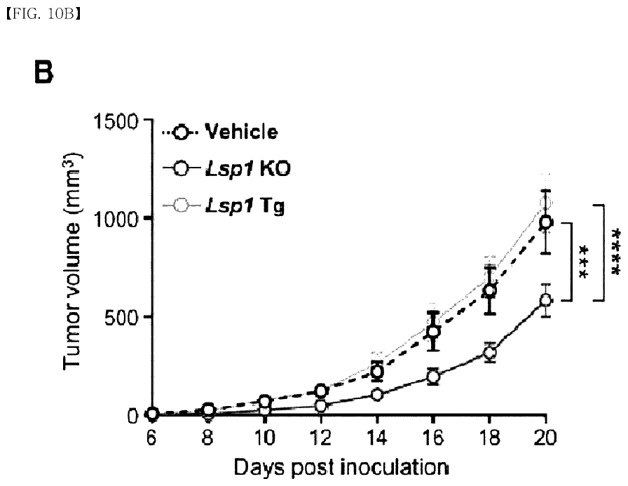

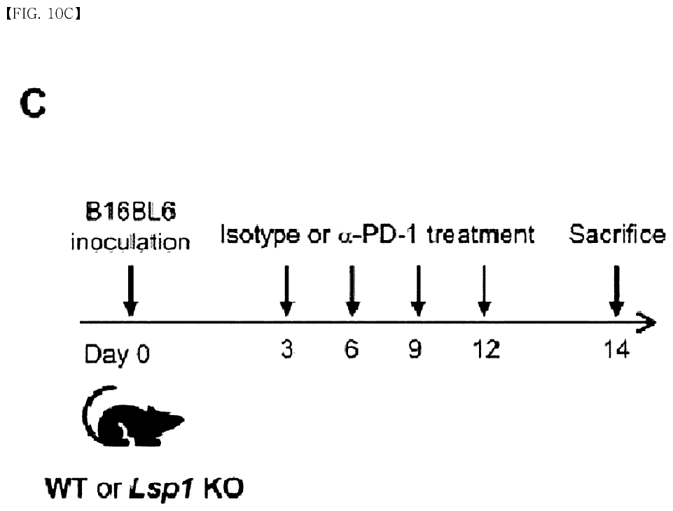

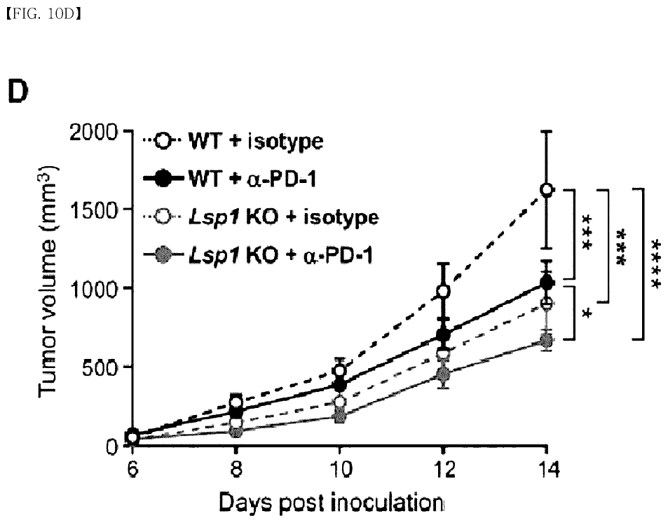

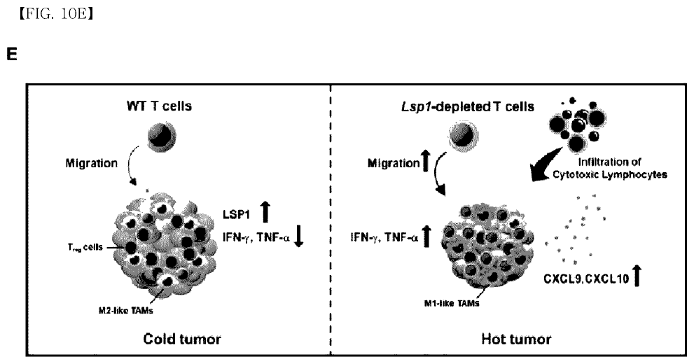

As a result, before the adoptive transfer experiment, Lsp1 KO T cells and Lsp1 Tg T cells had similar CD4/CD8 ratios in CD3⁺ T cells (FIG. 10A), and after the adoptive transfer experiment, Lsp1-deficient T cells more strongly inhibited tumor progression in Rag1 KO mice with adoptive transfer of B16 melanoma compared to Lsp1-overexpressed T cells and a vehicle alone (FIG. 10B), and thus, this verified that Lsp1 deficiency in T cells specifically mediated anti-tumor effects.

5-2. Confirmation of Effect of Lsp1-Deficiency by Combined Treatment with Anti-PD-1 Antibody

Since anti-PD-1 blockade, an immune checkpoint inhibitor that has been used as immunotherapy for patients with various advanced cancers, including melanoma, and the approach of the present invention had different anti-tumor mechanisms (suppressive immune checkpoint blockade/T cell trafficking), it was confirmed whether the anti-tumor effect caused by Lsp1 deficiency was affected by co-administration with anti-PD-1 Ab. Specifically, for anti-PD-1 blocking therapy, 5×10⁵ B16 melanomas were inoculated subcutaneously into the right flanks of WT mice and Lsp1 KO mice (day 0), and after 3, 6, 9, and 12 days, administered intraperitoneally with 10 mg/kg of anti-PD-1 antibody (RMP1-14, Bio X cell, Lebanon, NH) or its isotype control antibody, rat IgG2a isotype control (2A3, Bio X cell) (FIG. 10C).

As a result, when the tumor volume reached 1500 mm³ in WT mice treated with isotype control Ab, in WT mice treated with anti-PD-1 Ab, the tumor volume was 1000 mm³, and in Lsp1 KO mice treated with anti-PD-1 Ab, the tumor volume was 500 mm³ (FIG. 10D). That is, in WT mice treated with anti-PD-1 Ab, the growth of melanoma was significantly reduced, and particularly, Lsp1 KO mice treated with anti-PD-1 blockade showed a more significant anti-tumor effect than WT mice with or without anti-PD-1 antibody (FIG. 10D), and thus, it may be seen that the anti-tumor effect caused by Lsp1 deficiency is maintained regardless of anti-PD-1 antibody treatment.

As a result, it is suggested that genetic ablation of Lsp1 in T cells is a useful strategy to improve the therapeutic efficacy of the immune checkpoint inhibitor including anti-PD-1 Ab against melanoma (FIG. 10E).

1. A method for preventing or treating cancer comprising administering a pharmaceutical composition comprising a Leukocyte-specific protein 1 (LSP1) expression inhibitor as an active ingredient to a subject in need thereof.

2. The method of claim 1, wherein the expression inhibitor is small interfering RNA (siRNA), small hairpin RNA (shRNA), miRNA, antisense oligonucleotide, or nucleic acid aptamer.

3. The method of claim 1, wherein the expression inhibitor is a T cell-specific LSP1 expression inhibitor.

4. The method of claim 1, wherein the expression inhibitor comprises crRNA (CRISPR RNA) comprising a nuclease-deactivated Cas12a or Cas9 protein, a direct repeat (DR), and a protospacer complementary to an LSP1 gene.

5. The method of of claim 1, wherein the cancer is solid cancer.

6. The method of claim 1, wherein the composition increases the number and distribution frequency of T cells.

7. The method of of claim 1, wherein the composition increases the infiltration of T cells into tumors.

8. The method of claim 1, wherein the composition increases TNF-α and IFN-γ expression and secretion.

9. The method of claim 1, further comprising: an immune checkpoint inhibitor.

10. A method for preventing or treating cancer comprising administering a pharmaceutical composition comprising LSP1 gene-deleted T cells as an active ingredient to a subject in need thereof.

11. The method of claim 10, wherein the T cells are LSP1-deleted chimeric antigen receptor (CAR)-T cells.

12. The method of claim 11, wherein the CAR comprises a cancer cell antigen-binding domain, a transmembrane domain, and an intracellular signaling domain.

13. The method of claim 10, further comprising:
wherein the pharmaceutical composition further comprises an immune checkpoint inhibitor.

14. The method of of claim 13, wherein the immune checkpoint inhibitor is a PD-1 inhibitor or a CTLA-4 inhibitor.

15-16. (canceled)
